(19) United States
(12) Patent Application Publication
(10) Pub. No.: US 2025/0345300 A1
(43) Pub. Date: Nov. 13, 2025

(54) DEVICES, SYSTEMS AND METHODS FOR MEDICAMENT DELIVERY

(71) Applicant: MACH32 INC., Alberta (CA)

(72) Inventors: Marc CURIAL, Alberta (CA); Chris TERRIFF, Alberta (CA); Biju Isaac KORAVANKUDI, Alberta (CA); Will COMEAU, Alberta (CA); Ryley MCCONKEY, Alberta (CA)

(21) Appl. No.: 18/706,513

(22) PCT Filed: Nov. 2, 2022
(86) PCT No.: PCT/CA2022/051623

§ 371 Date: May 1, 2024

Related U.S. Application Data
(60) Provisional application No. 63/274,617, filed on Nov. 2, 2021.

(30) Foreign Application Priority Data
Jan. 14, 2022 (WO) PCT/CA2022/050057

(51) Int. Cl. A61K 31/195; A61M 5/20

(52) U.S. Cl. CPC A61K 31/195; A61M 5/20; A61M 2005/206; A61M 2005/2073

(57) ABSTRACT

A delivery device for the automatic injection of a dose of a fluid into the target site of a subject. The delivery device generally includes a lower body, an upper body that can be movably disposed over the lower body to activate the delivery device and an injection assembly operable for injecting and delivering a fluid to the target site. Methods for reducing for reducing or preventing hemorrhage, comprising intramuscularly administering an antifibrinolytic agent.

### CROSS-REFERENCE TO RELATED APPLICATIONS

This application claims the benefit of U.S. Provisional Patent Application Ser. No. 63/274,617 filed on Nov. 2, 2021, and PCT Application Serial No. PCT/CA2022/050057 filed on Jan. 14, 2022, the entire contents of both applications being hereby incorporated by reference herein.

### FIELD

The present disclosure generally relates to a delivery device. It also relates to methods for reducing or preventing hemorrhage comprising intramuscularly administering an antifibrinolytic agent.

### BACKGROUND

Trauma amounts to nearly 10% of worldwide deaths and is the leading cause of death under the age of 45. An essential element for treating many life-threatening emergencies, such as shock, trauma, cardiac arrest, drug overdoses, diabetic ketoacidosis, arrhythmias, burns, and status epilepticus, is rapid establishment of an intravenous (IV) line in order to administer drugs and fluids directly into a patient's vascular system. Whether in an ambulance by paramedics, in an emergency room by emergency specialists or on a battlefield by an Army medic, the goal is the same—quickly start an IV in order to administer lifesaving drugs and fluids. To a large degree, the ability to successfully treat most critical emergencies is dependent on the skill and luck of an operator in accomplishing vascular access. Doctors, nurses and paramedics can experience great difficulty in establishing IV access in many patients due to a variety of causes, such as patients with chronic disease or patients that may not have available IV sites due to anatomical scarcity of peripheral veins, obesity, extreme dehydration or previous IV drug use. A further complicating factor in achieving IV access occurs “in the field” e.g., at the scene of an accident, during military combat, or during ambulance transport where it is difficult to see the target and excessive motion makes accessing the venous system difficult.

Autoinjectors are devices that are designed to allow one the ability to self-administer a set dose of medication intramuscularly or subcutaneously. By providing a secondary route to the patient's systemic circulation that avoids obtaining an IV, autoinjectors circumvent many of the difficulties that IV's carry, especially in hectic situations described above, such as ambulance transport and military combat. Additionally, while IV lines must be placed by a trained healthcare professional, autoinjectors can be operated by members of the general public due to their simplicity and minimal risk of needlestick injuries or dosing errors.

In emergency situations involving massive hemorrhage and hemorrhagic shock, tranexamic acid (TXA) is considered a first-line medication. TXA is an antifibrinolytic drug that stops the breakdown of fibrin clots formed at the site of injury. In doing so, TXA causes a significant reduction in blood loss for the patient thereby decreasing patient mortality rates. TXA is most effective when given immediately after injury, but research has shown that TXA is only given to 3% of trauma victims within the first hour due to the difficulties of securing IV access. However, when TXA is given within an hour of the injury, it is shown to reduce deaths caused by hemorrhagic shock by one-third. Therefore, rapid TXA treatment that may be provided by the trauma patient themselves in the case that they do not have quick access to a trained healthcare professional is crucial to increase the chance of their survival. The ability for a trained healthcare professional or the trauma user to automatically inject TXA intramuscularly would significantly decrease the treatment time and increase the access to the drug without requiring an IV.

Known devices capable of accessing an IM site and/or administering drugs intramuscularly include, for example, the devices described in:
U.S. Pat. No. 10,556,067 which discloses a multiple use autoinjector that may be rearmed for multiple injections.
U.S. Pat. No. 8,961,463 which discloses an autoinjector allowing the automatic delivery of the first dose of a medicament, and the manual administration of the second.
U.S. Pat. No. 6,575,939 which discloses a single dose autoinjector comprising an inner and outer casing that are slidably arranged in relation to each other.
   It would be desirable to improve upon these state-of-the-art devices and provide a user-friendly, drug delivery system and preferably with an advanced safety mechanism to prevent needlestick injuries.

### SUMMARY

According to one embodiment, the present disclosure provides a delivery device operable to automatically inject and deliver a fluid to a target site of a subject. The delivery device comprises a lower body, an upper body configured to move relative to the lower body between a first upper body position and a second upper body position upon application of a force to the upper body and an injection assembly. The injection assembly comprises a fluid container configured to hold a fluid, the fluid container attached to the upper body and moveable with the upper body, a needle attached to the fluid container, the needle having a proximal end for insertion into the target site of the subject, wherein the delivery device is positionable such that the needle is inserted into the target site of the subject in response to the upper body moving from the first upper body position to the second upper body position, a plunger and a first releasable retainer member configured to release the plunger in response to the upper body reaching the second position. The first releasable retainer member releasing the plunger causes the plunger to urge the fluid out of the fluid container and into the needle to inject the fluid into the target site through the proximal end of the needle when the proximal end of the needle is inserted into the target site.

According to another embodiment, the present disclosure provides a delivery device operable to automatically inject and deliver a fluid to a target site of a subject. The delivery device comprises an injection assembly. The injection assembly comprises a fluid container configured to hold a fluid and having an outlet, a plunger moveable relative to the fluid container between a first plunger position and a second plunger position, a needle attached to the fluid container, in fluid communication with the outlet of the fluid container, and having a proximal end configured for insertion into the target site of the subject and a first releasable retainer member configured to secure the plunger in the first plunger position and release the plunger to cause the plunger to move to the second plunger position, wherein the first releasable retainer member releasing the plunger causes the plunger to urge the fluid out of the fluid container and into the needle to inject the fluid into the target site through the proximal end of the needle when the proximal end of the needle is inserted into the target site. The first releasable retainer member is in engagement with the plunger through at least interlocking threads when the plunger is in the first plunger position.

According to another embodiment, the present disclosure provides a delivery device comprising a fluid container, a fluid in the fluid container, wherein the fluid comprises an antifibrinolytic agent and a needle attached to the fluid container and having a proximal end for insertion into a target site of a subject. The delivery device is configured to inject and deliver the antifibrinolytic agent in a dose of about 0.1 g to about 30 g at a rate of no less than 50 mg/s to a target site.

According to another embodiment, the present disclosure provides a method for reducing or preventing hemorrhage, comprising intramuscularly administering an antifibrinolytic agent in a dose of about 0.1 g to about 30 g at a rate of no less than 50 mg/s.

According to another embodiment, the present disclosure provides a method for reducing or preventing hemorrhage, comprising intramuscularly administering an antifibrinolytic agent in a dose of about 0.1 g to about 30 g in about 1 mL to about 20 mL at a rate of no less than 0.1 mL/s.

According to another embodiment, the present disclosure provides a method for reducing or preventing hemorrhage, comprising intramuscularly administering an antifibrinolytic agent in a dose of about 0.1 g to about 30 g in about 1 mL to about 20 mL at a fluid velocity of no less than 0.2 m/s.

According to another embodiment, the present disclosure provides a method for reducing or preventing hemorrhage, comprising intramuscularly administering an antifibrinolytic agent in a dose of about 0.1 g to about 30 g, wherein the antifibrinolytic agent is administered within about 15 seconds.

According to another embodiment, the present disclosure provides a method for reducing or preventing hemorrhage, comprising intramuscularly administering an antifibrinolytic agent with an autoinjector in a dose of about 0.1 g to about 30 g.

According to another embodiment, the present disclosure provides the use of an antifibrinolytic agent for reducing or preventing hemorrhage, wherein the antifibrinolytic agent is for intramuscular administration in a dose of about 0.1 g to about 30 g at a rate of no less than 50 mg/s.

According to another embodiment, the present disclosure provides the use of an antifibrinolytic agent for reducing or preventing hemorrhage, wherein the antifibrinolytic agent is for intramuscular administration in a dose of about 0.1 g to about 30 g in about 1 mL to about 20 mL at a rate of no less than 0.1 mL/s.

According to another embodiment, the present disclosure provides the use of an antifibrinolytic agent for reducing or preventing hemorrhage, wherein the antifibrinolytic agent is for intramuscular administration in a dose of about 0.1 g to about 30 g in about 1 mL to about 20 mL at a fluid velocity of no less than 0.2 m/s.

According to another embodiment, the present disclosure provides the use of an antifibrinolytic agent for reducing or preventing hemorrhage, wherein the antifibrinolytic agent is for intramuscular administration in a dose of about 0.1 g to about 30 g within about 15 seconds.

According to another embodiment, the present disclosure provides the use of an antifibrinolytic agent for reducing or preventing hemorrhage, wherein the antifibrinolytic agent is for intramuscular administration with an autoinjector in a dose of about 0.1 g to about 30 g.

According to another embodiment, the present disclosure provides an antifibrinolytic agent for use in reducing or preventing hemorrhage, wherein the antifibrinolytic agent is for intramuscular administration in a dose of about 0.1 g to about 30 g at a rate of no less than 50 mg/s.

According to another embodiment, the present disclosure provides an antifibrinolytic agent for use in reducing or preventing hemorrhage, wherein the antifibrinolytic agent is for intramuscular administration in a dose of about 0.1 g to about 30 g in about 1 mL to about 20 mL at a rate of no less than 0.1 mL/s.

According to another embodiment, the present disclosure provides an antifibrinolytic agent for use in reducing or preventing hemorrhage, wherein the antifibrinolytic agent is for intramuscular administration in a dose of about 0.1 g to about 30 g in about 1 mL to about 20 mL at a fluid velocity of no less than 0.2 m/s.

According to another embodiment, the present disclosure provides an antifibrinolytic agent for use in reducing or preventing hemorrhage, wherein the antifibrinolytic agent is for intramuscular administration in a dose of about 0.1 g to about 30 g within about 15 seconds.

According to another embodiment, the present disclosure provides an antifibrinolytic agent for use in reducing or preventing hemorrhage, wherein the antifibrinolytic agent is for intramuscular administration with an autoinjector in a dose of about 0.1 g to about 30 g.

### BRIEF DESCRIPTION OF THE DRAWINGS

FIG. 1A is a cross-sectional view of a delivery device according to one embodiment in a first configuration;

FIG. 1B is a cross-sectional view of the delivery device of FIG. 1A in a second configuration;

FIG. 1C is a cross-sectional view of the delivery device of FIG. 1A in a third configuration;

FIG. 1D is a cross-section view of the delivery device of FIG. 1A in a fourth configuration;

FIG. 2 is an exploded view of a delivery device according to an embodiment of the present disclosure;

FIG. 3 is a cross-sectional view of the delivery device of FIG. 2 in a first configuration;

FIG. 4 is a cross-sectional view of the delivery device of FIG. 2 in a second configuration;

FIG. 5 is a cross-sectional view of the delivery device of FIG. 2 in a third configuration;

FIG. 6 is a cross-sectional view of the delivery device of FIG. 2 in a fourth configuration;

FIG. 7 is a perspective view of the delivery device of FIG. 2 in a first configuration;

FIGS. 8A-D are perspective views of a delivery device according to an embodiment of the present disclosure;

FIGS. 8E and 8F are cross-sectional views of the delivery device of FIGS. 8A-D with the driver at the first driver position;

FIG. 8G is a cross-sectional view of the delivery device of FIGS. 8A-D, prior to removal of the safety lock;

FIG. 8H is a cross-sectional view of the delivery device of FIGS. 8A-D with the driver at the second driver position; and

FIG. 9 is a perspective view of the safety lock of the delivery device of FIGS. 8A to 8H.

### DETAILED DESCRIPTION

The following terms shall have the following meanings:

The term “comprising” and derivatives thereof are not intended to exclude the presence of any additional component, step or procedure, whether or not the same is disclosed herein. In contrast, the term, “consisting essentially of” if appearing herein, excludes from the scope of any succeeding recitation any other component, step or procedure, except those that are not essential to operability and the term “consisting of”, if used, excludes any component, step or procedure not specifically delineated or listed. The term “or”, unless stated otherwise, refers to the listed members individually as well as in any combination.

The articles “a” and “an” are used herein to refer to one or to more than one (i.e., to at least one) of the grammatical objects of the article. By way of example, “a container” means one container or more than one container. The phrases “in one embodiment”, “according to one embodiment” and the like generally mean the particular feature, structure, or characteristic following the phrase is included in at least one embodiment of the present disclosure, and may be included in more than one embodiment of the present disclosure. Importantly, such phrases do not necessarily refer to the same aspect. If the specification states a component or feature “may”, “can”, “could”, or “might” be included or have a characteristic, that particular component or feature is not required to be included or have the characteristic.

As used in this specification and the appended claims, the words “proximal” and “distal” refer to directions closer to and away from, respectively, the desired position of injection and delivery of fluid of the delivery device. The words “upward”, “downward”, “upper”, “lower”, “right” and “left” are relative terms used to designate components and/or directions for convenience and are not intended to be limiting. For example, an upper part could be located below a lower part depending on the direction of view (and vice versa). The words “inward” and “outward” refer to directions toward and away from, respectively.

The term “intramuscular site” or “IM site” refers to a position where an injection of a fluid can be administered into any muscle of a subject, such as the deltoid, vastus lateralis, rectus femoris or the ventrogluteal and dorsogluteal areas.

The term “movably coupled” means that one member is directly or indirectly supported by another member to allow movement of the one member.

The term “operatively coupled” can refer to a direct or indirect coupling engagement between two or more structural component parts.

The term “fluid” includes any liquid, such as but not limited to, blood, water, saline solutions, IV solutions or plasma, or any mixture of liquids, particulate matter, medicament, dissolved medicament and/or drugs appropriate for injection into the target site of a subject.

The term “container” refers to a pharmaceutically acceptable container comprising a chamber suitable to house a fluid. Containers can include, but are not limited to vials, barrels, ampoules or bottles and in some embodiments are made of glass, plastic, composites, laminates or metal.

As used herein, a “subject” may be a human or non-human mammal. Non-human mammals include, for example, livestock and pets, such as ovine, bovine, porcine, canine and feline mammals. Preferably, the subject is a human and in some embodiments the operator and the subject are the same (i.e., the delivery device is a self-administering delivery device).

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

In one aspect, embodiments of the present disclosure are generally directed to a delivery device adapted to automatically inject a doses of a fluid into a target site, such as an IM site of a subject. In some embodiments, the delivery device is sized to be carried in compact spaces such as, but not limited to, military pouches and tactical vests.

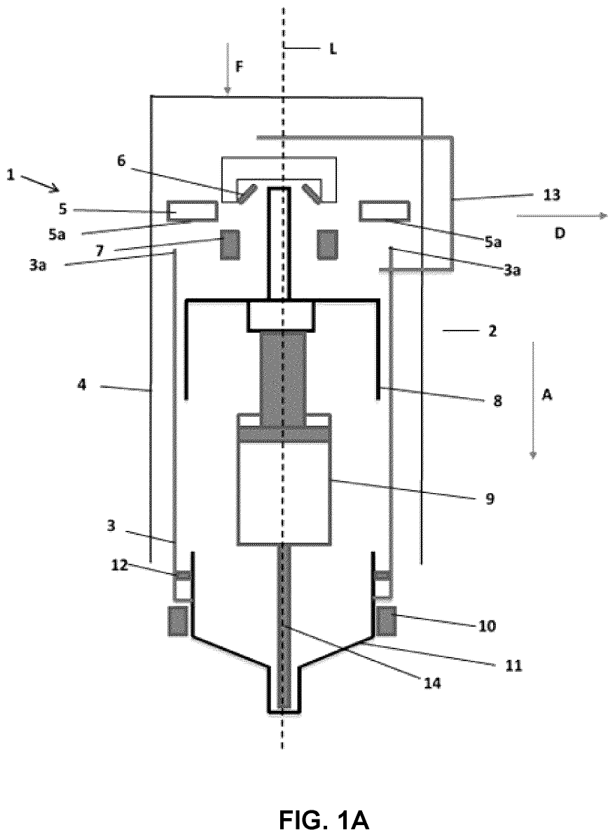

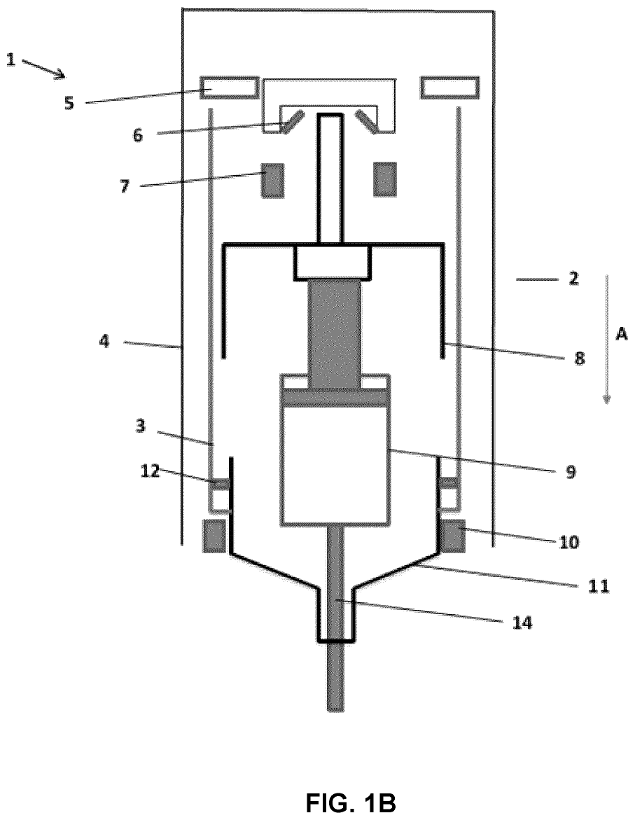

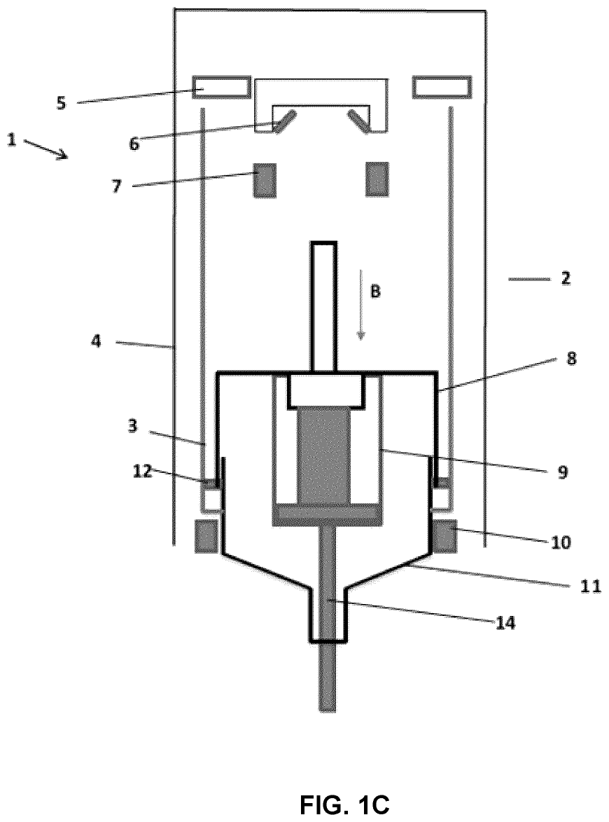

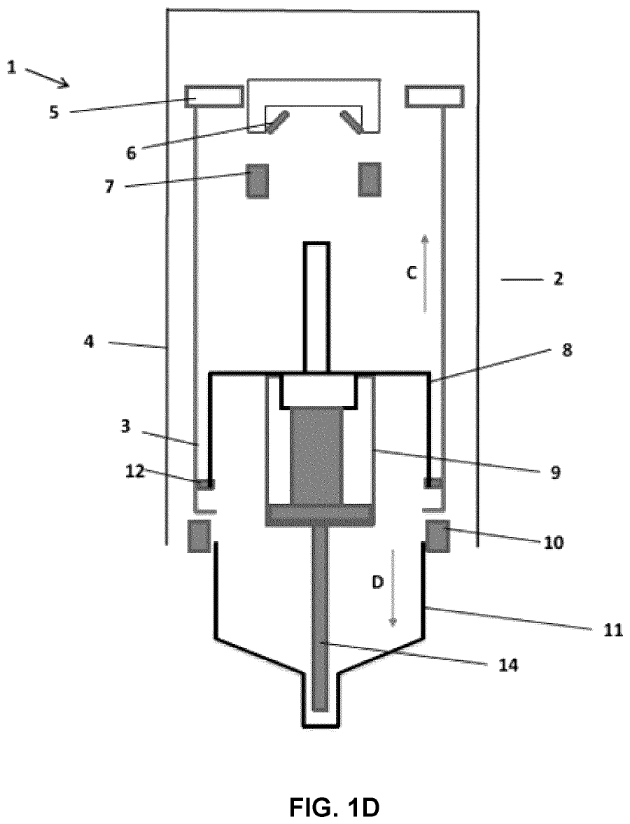

Turning now to the drawings and in particular, to FIGS. 1A-1D, a cross-sectional view of a delivery device 1 is shown according to one embodiment advancing sequentially from a first configuration to a second configuration, to a third configuration and to a fourth configuration, respectively. In general terms, the delivery device 1 includes an upper body 4 and a lower body which are both configured to house an injection assembly 2. Injection assembly 2 is configured to inject and deliver a fluid to the IM site of the subject. In some embodiments, the injection assembly is configured to inject and deliver at least 1 mL or at least 2 mL or at least 3 mL or at least 4 mL or at least 5 mL of the fluid to the IM site of the subject. Although one injection assembly is shown, the delivery device 1 may include one or more injection assemblies. The delivery device 1 also includes a lower body 3 which is movably coupled to the upper body 4. The upper body 4 is configured to be moveable relative to the lower body 3 in a proximal direction along the longitudinal axis L of delivery device 1 (FIG. 1A) upon application of a force F to the upper body 4.

The injection assembly includes a plunger 8 moveable between a first plunger position and second plunger position, a first releasable retainer member 6 configured to secure the plunger 8 in the first plunger position and release the plunger 8 after activation of the injection assembly 2, a first energy storage member 7 operable to release energy to the plunger 8 and displace the plunger 8 in the proximal direction from the first plunger position to the second plunger position, and a fluid container 9 having a distal end configured to receive the plunger 8 and a proximal end with an orifice or outlet. The orifice or outlet of container 9 may be in fluid communication with a needle 14. The plunger 8 is sized to be movably disposed inside the lower body 3. In some embodiments, the fluid comprises a medicament, such as, for example, tranexamic acid or any of the other medicaments further described below.

The injection assembly 2 may include a safety lock to prevent activation of injection assembly 2. The safety lock may be any suitable mechanism, such as a button or a pin. In the embodiment shown in FIGS. 1A-1D the safety lock is a safety pin 13 which needs to be removed before activating the injection assembly 2. When in the position shown in FIG. 1A, safety pin 13 may be engaged with upper body 4 and lower body 3, such as by insertion of a portion of safety pin 13 in openings of upper body 4 and lower body 3, to prevent movement of upper body 4 relative lower body 3. When safety pin 13 is removed, upper body 4 may be free to move relative to lower body 3.

The delivery device 1 also includes a driver 11 moveable relative to lower body 3 from a first driver position to a second driver position and having a proximal end and a distal end. The delivery device 1 also includes a second energy storage member 10 operable to release energy to the driver 11 and displace the driver 11 in a proximal direction when the plunger 8 reaches the second plunger position and detaches the driver 11 from lower body 3. The lower body 3 and the driver 11 are releasably coupled by second releasable retainer member 12, which is configured to secure driver 11 to lower body 3 and release driver 11 from lower body 3 when plunger 8 is located at the second plunger position.

The delivery device 1 can generally be operated by removing the safety pin 13 in the direction indicated by arrow D in FIG. 1A. The user may place the proximal end of driver 11 in contact to the target site and apply a force to the upper body 4 in the proximal direction. The upper body 4 moves in a proximal direction over lower body 3, such that a portion of needle 14 (which interconnected to upper body 4) extends from the proximal end of driver 11. The movement of upper body 4 in a proximal direction also activates the actuator 5. Activation of actuator 5 causes an automatic sequence of movements. First, activation of the actuator 5 causes the first releasable retainer member 6 to release plunger 8. The plunger 8 is then displaced in a proximal direction by the first energy storage member 7. Movement of the plunger 8 in the proximal direction will continue until reaching the second plunger position at the proximal end of the fluid container 9 (FIG. 1C). As plunger 8 moves proximally, the liquid is delivered from/urged out of fluid container 9 to needle 14, as will be explained in more detail below. The first energy storage member 7 is operable to continue to apply energy to the plunger 8 that is sufficient for plunger 8 to engage second releasable retainer member 12 such that driver 11 is released from lower body 3. The user may hold the device to the target site for a prescribed time period, which may be a time sufficient for the liquid to be delivered/urged out of the fluid container 9 to and through needle 14 for delivery to the target site. The prescribed time period may be selected based on factors such as the volume of fluid to be delivered and the configuration of device 1. After this prescribed time period, the user reduces the force F applied to the upper body 4. The second energy storage member 10 exerts a force on the lower body 3 in the distal direction (as indicated by arrow C in FIG. 1D) which in turn displaces upper body 4 in a distal direction which is sufficient, in combination with the reduced force F applied to the upper body 4 to displace delivery device 1 in a distal direction relative to the target site such that needle 14 is retracted from the target site. As this happens, driver 11 is displaced proximally by second energy storage member 10 relative to the needle and lower body 3 from its first driver position (FIG. 1C) to a second driver position (FIG. 1D). As the driver 11 is displaced with respect to the lower body 3, the needle 14 is retracted back from the target site and is enclosed within driver 11.

Referring to FIGS. 1A and 1B, the delivery device 1 is shown in a first configuration and a second configuration respectively. To move from the first configuration to second configuration, the upper body 4 is displaced over the lower body 3 in a proximal direction, as indicated by arrow A in FIGS. 1A and 1B, along a longitudinal axis L of the delivery device between a first upper body position (FIG. 1A) and a second upper body position (FIG. 1B). As illustrated, when the upper body 4 is displaced in a proximal direction from the first upper body position to the upper body second position, a distal portion 3a of the lower body 3 makes contact with a proximal portion 5a of actuator 5 (FIG. 1A), which activates actuator 5. The actuator 5 may be any suitable device for activating the delivery device 1, such as, for example, a handle, a lever, a push button, lock, a slidable button or a trigger.

Prior to activation of actuator 5, the plunger 8 is held relative to the upper body in the first plunger position by the first releasable retainer member 6. The first releasable retainer member 6 is configured to release the plunger 8 and release the first energy storage member 7 from its first configuration to second configuration wherein the first energy storage member 7 is able to release energy to the plunger 8 to displace plunger 8 when in the second configuration, after actuator 5 has been activated by movement of the upper body 4 in the proximal direction from the first actuator position (FIG. 1A) to the second actuator position (FIG. 1B). The first releasable retainer member 6 can be any suitable mechanism for releasably retaining the plunger 8 and releasing the first energy storage member 7, such as, for example, a mechanical linkage, a compressed ring, a spring-loaded rod, interlocking threads, a tensioned latch or tab or the like. In an embodiment, the interlocking threads are a helical male and female thread arrangement.

When the delivery device 1 is in the first configuration, the needle 14, which is in fluid communication with the fluid container 9 is housed within the driver 11. When the delivery device moves to the second configuration, a portion of needle 14 extends outside the lower body 3 (due to proximal movement of the upper body 4 and the interconnected needle 14 relative to lower body 3) for insertion into the target site of a subject at a desired depth. Thus, movement of the delivery device 1 from the first configuration to second configuration will extend the needle 14 outside of the lower body 3 for insertion into, for example, the IM site of the subject.

With reference to FIGS. 1B and 1C (which depicts the delivery device 1 in a third configuration), the plunger 8 is displaced in a proximal direction relative to upper body 4, as indicated by arrow B, along a longitudinal axis L of the delivery device 1 between a first plunger position (FIG. 1B) and second plunger position (FIG. 1C) by deployment of the first energy storage member 7. When the plunger 8 is in the first plunger position, a portion of the proximal end of the plunger 8 is positioned at the distal end of the fluid container 9. In this first plunger position, the plunger 8 is in fluid communication with the fluid contained within fluid container 9. When the plunger 8 is displaced (or advanced) to the second plunger position, the plunger 8 is advanced from the distal end of the fluid container 9 to the proximal end of the fluid container 9. In this manner, as the plunger 8 is displaced (or advanced) between the first and second plunger positions, fluid is conveyed/urged out from within fluid container 9 through the needle 14 and into the IM site of the subject. Thus, movement of the delivery device 1 from the second configuration to third configuration delivers fluid into the IM site of the subject.

With reference now to FIG. 1D (which depicts the delivery device 1 in a fourth configuration), prior to the plunger 8 reaching the second plunger position, the driver 11 is held in a secure position to the lower body 3 by the second releasable retainer member 12. The second releasable retainer member 12 is configured to release the driver 11 once the plunger 8 reaches the second plunger position and release the second energy storage member 10 from its first configuration (FIG. 1C) to its second configuration (FIG. 1D), wherein the second energy storage member 10 is able to release energy to the driver 11 to displace the driver 11 when in the second configuration. The second releasable retainer member 12 can be any suitable mechanism for releasably retaining the driver 11 and deploying the second energy storage member 10, such as, for example, a mechanical linkage, a compressed ring, a spring-loaded rod, a tensioned latch or tab or the like.

Upon release of the driver 11 by the second releasable retainer member 12, the second energy storage member 10 is also released from its first configuration to its second configuration. Energy released from the second energy storage member 10 displaces the lower body 3 and the upper body 4 in the distal direction, which is sufficient, in combination with the reduced force F applied to the upper body 4 to displace delivery device 1 in a distal direction relative to the target site such that needle 14 is retracted from the target site. As this happens the driver 11 is displaced by second energy storage member 10 proximally, as indicated by arrow D in FIG. 1D, along the longitudinal axis L of delivery device 1 from its first driver position (FIG. 1C) to its second driver position (FIG. 1D) where the needle 14 is fully within driver 11.

The first and second energy storage members 7 and 10 each independently can be any device for storing energy. Thus, one or both of the first and second energy storage members 7 and 10 may be a mechanical energy storage member, such as a spring, a device containing compressed gas, a device containing a vapor pressure-based propellant or something similar or an electrical energy storage member, such as a battery, a capacitor, a magnetic energy storage member or something similar In yet other embodiments, one or more of the first and second energy storage members 7 and 10 can be a chemical energy storage member, such as a container containing two substances that, when mixed, react to produce energy.

The first energy storage member 7 moves within the upper body 4 along axis L between a first configuration (FIGS. 1A and 1B) and a second configuration (FIG. 1C). When the first energy storage member 7 is in its first configuration, it has a first potential energy. When the first energy storage member 7 is in its second configuration, it has a second potential energy that is less than the first potential energy. The first energy storage member 7 is operably coupled to the plunger 8 such that when the first energy storage member 7 moves from its first configuration to its second configuration, it converts at least a portion of its first potential energy into kinetic energy to displace the plunger 8 in the proximal direction from the first plunger position to the second plunger position. Said another way, the movement of the first energy storage member 7 from its first configuration to its second configuration results in the release of energy that acts upon the plunger 8 to move the plunger 8 from the first plunger position (FIGS. 1A and 1B) to the second plunger position (FIG. 1C) and thereby dispense fluid contained within the fluid container 9. Moreover, the energy released is also sufficient to activate the second releasable retainer member 12, thereby detaching the driver 11 from lower body 3.

Similarly, the second energy storage member 10 can be moved within the driver 11 and lower body 3 along L between a first configuration (FIG. 1C) and a second configuration (FIG. 1D). When the second energy storage member 10 is in its first configuration, it also has a first potential energy. When the second energy storage member 10 is in its second configuration, it has a second potential energy that is less than the first potential energy. The second energy storage member 10 is operably coupled to the driver 11 such that when the second energy storage member 10 moves from its first configuration to its second configuration, it converts at least a portion of its first potential energy into kinetic energy to displace (in combination with the reduced force F applied to the upper body 4) the lower body 3 and upper body 4 distally and displace the driver 11 in the proximal direction from the first driver position to the second driver position. Upper body 4 and needle 14, which is secured to the upper body 4 also moves distally at the same time.

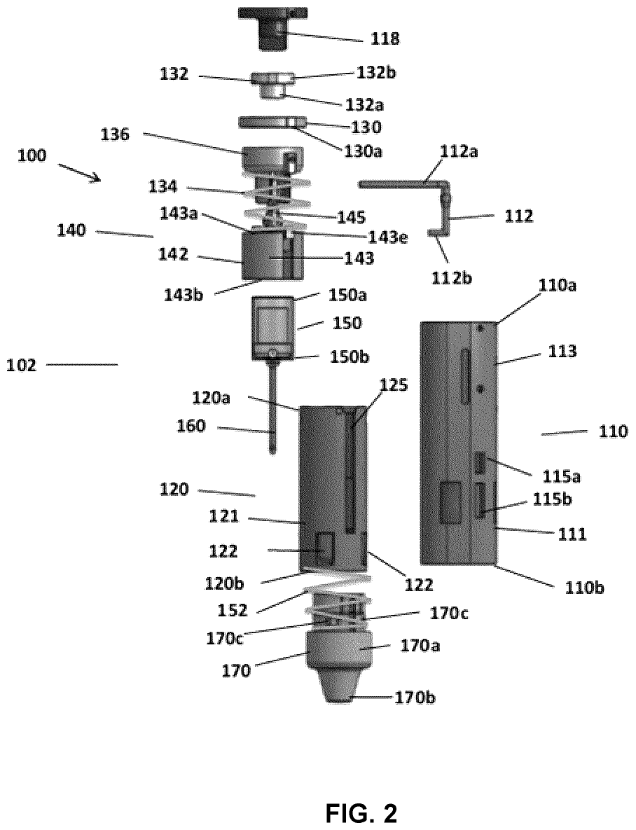

With reference now to FIGS. 2-7, a delivery device according to an embodiment is shown and generally designated by reference numeral 100. The delivery device 100 generally includes an upper body 110 and a lower body 120 movably coupled to the upper body 110. The upper body 110 is generally shaped and dimensioned to fit within an operator's hand and includes a distal end 110a, a proximal end 110b and an exterior surface 111. The upper body 110 may be a unitary structure (i.e., one-piece) that defines the exterior surface 111, or it may include a plurality of layers with different layers defining the exterior surface 111.

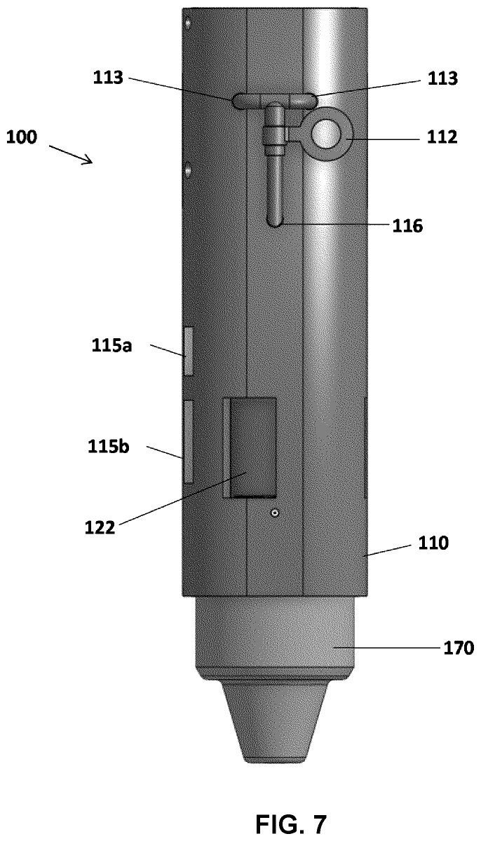

The upper body 110 may further include a safety pin 112 movably coupled thereto which may be operated by the operator. The safety pin 112 is adapted and configured to prevent the premature or accidental activation of the delivery device 100. In one embodiment, the safety pin 112 is a pull pin arrangement, such as shown in FIG. 2 but in other embodiments may also be a switch, a button, or similar mechanism to prevent accidental activation. In the embodiment shown in FIGS. 2-7, safety pin 112 may be inserted into a pair of upper openings 113 and a lower opening 116 (FIG. 7).

In some embodiments, the upper body 110 may be rigid. According to other embodiments, the upper body 110 may be flexible, whether according to the nature of the material that defines the upper body 110 or according to the nature of the structure of the upper body 110. The upper body 110 may be made of glass, metal, or polymer, for example. In particular, polymer versions may be made of polycarbonate, polypropylene, polyethylene (such as high density polyethylene), polytetrafluoroethylene, cyclic olefin polymer, cyclic olefin copolymer, crystal zenith olefinic polymer, nylon, or engineering resins. As to flexible versions of the upper body 110, butyl rubber, silicon-based rubber, latex-based rubber, coated rubber, as well as multi-layer polymer films, such as polyethylene (such as low density polyethylene) and polypropylene, may be used.

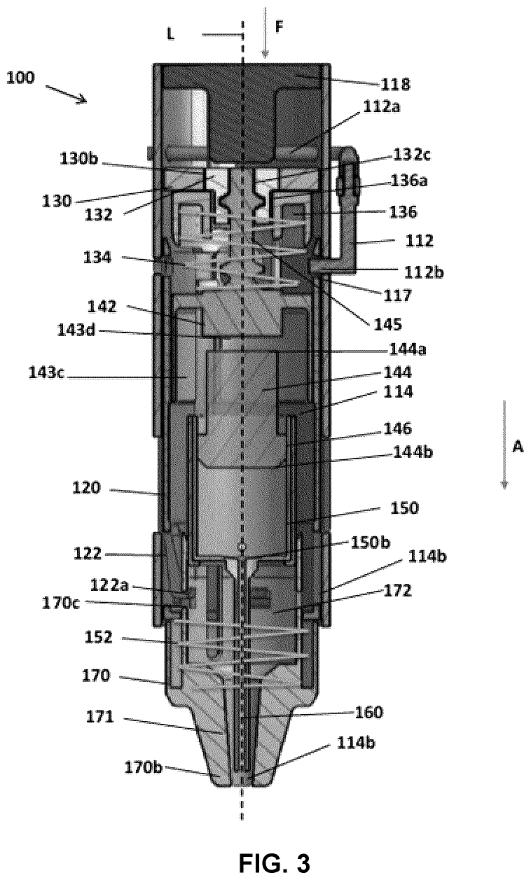

The device 100 has a chamber 114 (FIG. 3), sized and configured to receive injection assembly 102. Although one injection assembly is shown, the upper body 110 may include more than one chambers for holding more than one injection assembly if desired. Chamber 114 includes openings at both the distal end 110a and the proximal end 110b of upper body 110. Such openings may include a breakable or removable seal or cover for sterilization purposes prior to use. For example, a plug 118 may be disposed at the distal end 110a of chamber 114. In some embodiments plug 118 may be permanently attached to upper body 110 or formed as an integral part of upper body 110. In other embodiments, plug 118 may be removable from upper body 110.

A fluid container 150 may be located within the chamber 114 and secured to and is movable with the upper body 110. In some embodiments, fluid container 150 may be secured to upper body by an interference fit; adhesive, fasteners (such as screws, rivets, clips), interlocking structural elements or any suitable means that restricts the relative motion of fluid container 150 relative to upper body 110. In an embodiment, fluid container 150 may be secured to upper body 110 through an interference fit in combination with a suitable adhesive.

The lower body 120 includes a distal end 120a and a proximal end 120b and is generally shaped and dimensioned such that upper body 110 may move relative to lower body 120 in a proximal direction along a longitudinal axis L of delivery device 100 (FIG. 3) from a first upper body position to a second upper body position upon application of user applied force F at the distal end 110a or along the exterior surface 111 of the upper body 110 directed in a proximal direction, generally towards the target IM site. The lower body 120 includes an exterior surface 121. The lower body 120 may be a unitary structure (i.e., one-piece) that defines the exterior surface 121, or it may include a plurality of layers with different layers defining the exterior surface 121. The lower body 120 may further include a releasable retainer member 122 releasably coupled to a driver 170.

In some embodiments, upper body 110 may feature one or more elongated windows sized and configured to allow the operator to view the contents of lower body 120. The windows may be any suitable shape for viewing the contents, such as, but not limited to, an arrow, rectangle or a long oval. The windows may comprise a clear material, such as a translucent or transparent material to maintain the sterility of the delivery device 100 whilst allowing the operator to view the contents of upper body 110. For example, upper body 110 has elongated windows 115a and 115b which are sized and configured to allow the operator to view the contents of fluid container 150 of injection assembly 102 (FIG. 2). Windows 115a and 115b may align with a longitudinally extending slot 125 in the lower body 120 such that at least a portion of fluid container 150 may be in view. This may allow visualization of the fluid prior to delivery and allow a user to confirm that the fluid has been delivered.

With continued reference to FIGS. 2 and 3, the delivery device 100 as shown includes only one injection assembly 102 and in some embodiments there may a second injection assembly. In such embodiments the components for additional injection assemblies and their arrangement and operation would be similar to injection assembly 102 and accordingly only one injection assembly 102 will be described below.

The injection assembly 102 includes an actuator 130, a first releasable retainer member 132, a first energy storage member 134, a plunger 140, and a fluid container 150.

First releasable retainer member 132 may be seated in a collar 136 secured to the upper body 110. The first releasable retainer member 132 includes a generally circular proximal portion 132a, a distal portion 132b and a threaded opening 132c (FIGS. 2 and 3). The collar 136 has a centrally located opening 136a into which the proximal end 132a of the first releasable retainer member 132 is received, such that member 132 may be rotatable relative to collar 136 within the opening 136a.

The actuator 130 may be generally ring shaped and includes a centrally located opening 130b for engaging the outer edge of the first releasable retainer member 132 such that actuator 130 interlocks with first releasable retainer member 132. Actuator 130 is moveable relative to the first releasable retainer member 132. When actuator 130 is in the first actuator position, as shown in FIG. 3, actuator 130 is positioned directly above collar 136 and engages distal portion 132b of first releasable retainer member 132 such that rotational movement of first releasable retainer member is prevented.

The plunger 140 may include a plunger rod 142. The plunger rod 142 is shown having a cylindrical body 143 with a distal end 143a, a proximal end 143b an inner cavity 143c. The plunger rod 142 may include a plunger piston contact member 143d within cavity 143c (FIG. 3). The plunger rod 142 and first energy storage member 134 are operatively coupled at the distal end 143a of the plunger rod 142. The plunger rod 142 is configured to be slidably movable within the lower body 120 in a longitudinal axis L shown in FIG. 3. An outwardly projecting tab 143e (FIG. 2) on the outer surface of the cylindrical body 143 is received within the longitudinally extending slot 125 in the lower body 120. The slot 125 may guide plunger rod 142 during longitudinal movement of the plunger rod 142 within the lower body 120.

The plunger rod 142 may also include a threaded member 145 extending in a distal direction from the distal end 143a of the cylindrical body 143. When the plunger rod 142, first releasable retainer member 132 and actuator 130 are in their first positions (as shown in FIG. 3), a distal portion of the threaded member 145 is received within the threaded opening 132c of the first releasable retainer member 132 and actuator 130 is also in engagement with the first releasable retainer member 132.

When first energy storage member 134 is in its first position (FIG. 3) it will be exerting a force upon plunger rod 142 in a proximal direction which, acting through the threaded connection between plunger rod 142 and first releasable retainer member 132, will bias member 132 to rotate. However, rotation of first releasable retainer member 132 is prevented by engagement with actuator 130, which is in turn prevented from rotational movement by engagement of the tab 130a with the longitudinally extending slot (not shown) on the interior surface of the upper body 110. Additionally, plunger rod 142 is prevented from rotational movement by engagement of outwardly projecting tab 143e within the longitudinally extending slot 125 in the lower body 120. As such, plunger rod 142 cannot disengage from first releasable retainer member 132 whilst actuator 130 is in the first actuator position.

With this arrangement, when delivery device 100 is in the first configuration, movement of the actuator 130 in the distal direction is limited due to the engagement with the upper portion 112a of safety pin 112, which in turn limits the rotation of the first releasable retainer member 132, which in turn limits movement of the plunger 140 in the proximal direction. Thus, both the plunger 140 and actuator 130 are retained before use.

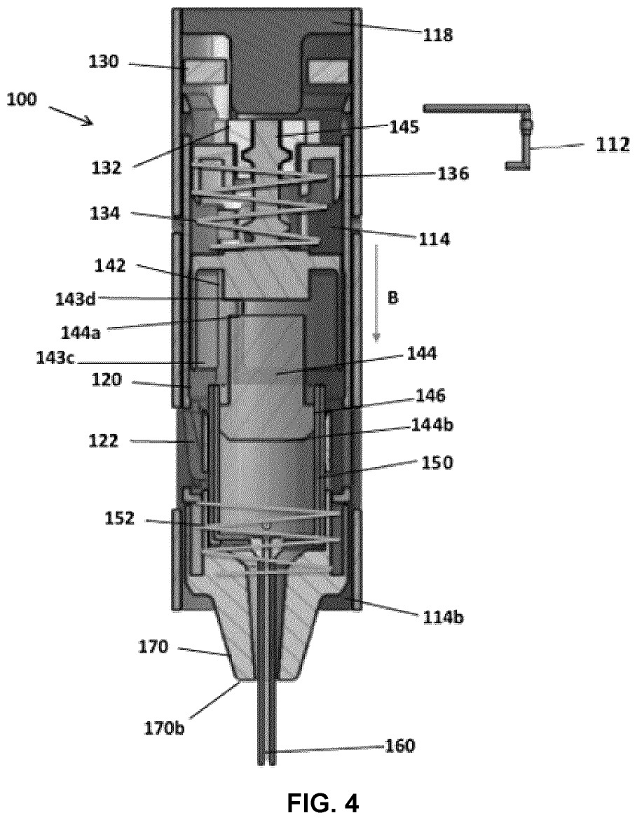

Once safety pin 112 is removed from delivery device 100, actuator 130 is lightly restrained from motion in the distal direction by frictional forces with first releasable retainer member 132. The movement of the actuator 130 in the proximal direction is restricted by the distal surface of collar 136 and may only move distally from the first actuator position. This is caused by movement of lower body 120 in a distal direction from the first actuator position (FIG. 3) to the second actuator position (FIG. 4), whereby the distal end 120a of the lower body 120 will contact the proximal surface of the actuator 130, displacing actuator 130 in a distal direction from the first actuator position and thereby disengaging the actuator 130 from the first releasable retainer member 132. As actuator 130 disengages with the first releasable retainer member 132, the member 132 is free to rotate about longitudinal axis L relative to collar 136, such that threaded member 145 and thereby plunger rod 142 is released to disengage from member 132 and is able to move in a proximal direction from the first plunger position to the second plunger position.

With reference to FIGS. 2 and 3, distal movement of actuator 130 is prevented by safety pin 112. When safety pin 112 is inserted, an upper portion 112a of the safety pin 112 extends through the upper openings 113 within upper body 110 above actuator 130, preventing movement of actuator 130 in a distal direction, whilst a lower portion 112b of the safety pin 112 extends through the lower opening 116 in the upper body 110 and also an opening 117 in the lower body 120. Through this arrangement, when the lower portion 112b of the safety pin is disposed within the openings 116 and 117, the upper body 110 and the lower body 120 are unable to move relative to each other.

Once safety pin 112 is removed actuator 130 may move distally within chamber 114 from the first actuator position as shown in FIG. 3 to the second actuator position shown in FIG. 4 such that actuator 130 disengages with first releasable retainer member 132. The actuator 130 is advanced in the distal direction by the movement of the lower body 120. The actuator 130 may include a tab 130a on the exterior circumference which engages a longitudinally extending slot (not shown) on the interior surface of the upper body 110, which may act to prevent rotation of actuator (130 about longitudinal axis L) relative to first upper body 110. As will be described in detail below, such movement by the actuator 130 from the first actuator position to the second actuator position activates the delivery device 100 to cause a sequence of movements of the injection assembly 102 that subsequently leads to the injection and delivery of fluid contained within the injection assembly 102.

In some embodiments, the injection assembly 102 may further include a safety activation mechanism operatively coupled with actuator 130 which prevents the actuator 130 from moving in a distal direction thus preventing the first releasable retainer member 132 being activated and releasing the plunger 140 from engagement until the safety activation mechanism is activated. Thus, safety activation mechanism is configured to prevent the premature or accidental deployment of the delivery device 100. The safety activation mechanism can be comprised of any suitable mechanism, such as an energy storage member described above, a button, a pin, or similar mechanism. In such embodiments, regardless of whether the safety pin 112 has been released from engagement, the safety activation mechanism will prevent the actuator 130 from activation. The safety activation mechanism therefore acts as a second safety mechanism and will prevent the premature or accidental activation of the delivery device, irrespective of the release of safety pin 112. The safety pin 112 may also be releasably coupled to the lower body 120 and the upper body 110, preventing movement of the lower body 120 relative to the upper body 110, thus preventing displacement of the actuator 130 from the first actuator position.

The injection assembly 102 also includes a fluid container 150 and a needle 160. The fluid container 150 is configured to hold a fluid. The fluid container 150 includes an open distal end 150a and a proximal end 150b comprising an outlet or orifice that is fluidly coupled to needle 160, such as by a luer connector, threads, a snap-fit, a latch, a lock, a friction fit coupling, an adhesive or any other suitable coupling features (not shown). The fluid container 150 may have a length of between about 10 millimeters (mm) to about 100 mm. In an embodiment the fluid container has a length of about 77 mm. The distal end 150a and proximal end 150b may have a diameter of between about 10 mm to about 25 mm. The orifice at the proximal end 150b may have a diameter of between about 0.05 mm to about 1.6 mm.

The fluid container 150 defines an internal volume configured to house a fluid. In some embodiments the fluid container 150 may be configured to hold an amount of fluid in the range of about 1 milliliter (mL) to about 20 mL, or about 2 mL to about 15 mL, or about 3 mL to about 10 mL, or about 4 mL to about 6 mL. The fluid may comprise a medicament such as, but not limited to, an analgesic, anti-inflammatory agent, anthelmintic, anti-arrhythmic agent, antibiotic (including penicillin), anticoagulant, antidepressant, antidiabetic agent, antiepileptic, antihistamine, antihypertensive agent, antimuscarinic agent, antimycobacterial agent, antineoplastic agent, antifibrinolytic, immunosuppressant, antithyroid agent, antiviral agent, anxiolytic sedative (hypnotics and neuroleptics), astringent, beta-adrenoceptor blocking agent, blood product and substitutes, cardiac inotropic agent, corticosteroid, cough suppressant (expectorants and mucolytics), diagnostic agent, diuretic, dopaminergic (antiparkinsonian agents), haemostatic, immunological agent, lipid regulating agent, muscle relaxant, parasympathomimetic, parathyroid calcitonin and biphosphonate, prostaglandin, radiopharmaceutical, sex hormone (including steroids), anti-allergic agent, stimulant and anorexic, sympathomimetic, thrombolytic, thyroid agent, PDE IV inhibitor, NK3 inhibitor, ppar agent, NK-2 inhibitor, CSBP/RK/p38 inhibitor, antipsychotic, vasodilator, xanthine, and antidote (e.g., to a toxin or to a biological, chemical, or radiological weapon).

The injection assembly 102 also includes a first energy storage member 134. In the embodiment shown in FIG. 3, the first energy storage member 134 is a mechanical energy storage member comprising a spring, such as, for example, a helical, compression, extension, torsion, constant, variable, variable stiffness or any other type of spring having a spring constant ranging between about 1 N/m to about 3000 N/m. In some embodiments the spring has a spring constant about 1680 N/m. The first energy storage member 134 is operatively coupled to the plunger 140 and releases energy to the plunger 140 to displace the plunger 140 in the proximal direction when the first energy storage member 134 moves from its first configuration to second configuration after the actuator 130 has been activated.

As shown in FIGS. 2 and 3, the plunger 140, also includes a plunger piston 144. In some embodiments, the plunger 140 may be formed as a single piece or as modular components. The modular components may be affixed to one another or located adjacently but not connected, so as to move together. The plunger 140 is sized and configured to be movably disposed within the fluid container 150 when it moves between the first plunger position and second plunger position.

The plunger piston 144 has a distal end 144a and a proximal end 144b which is configured to be in fluid communication with the fluid disposed within the internal volume defined by the fluid container 150. In some embodiments, plunger piston 144 may be supplied with (and installed in) fluid container 150.

In some embodiments, plunger piston contact member 143d of the plunger rod 142 can be coupled to and/or in contact with the distal end 144a of the plunger tip 144. In other embodiments, there may be a gap between plunger piston contact member 143d and the distal end 144a of the plunger piston 144 (as shown in FIG. 3). In some embodiments, another component may be positioned between the plunger piston contact member 143d and the distal end 144a of plunger piston 144. The component may be made from a compressible material, such as a foam and may minimize or dampen any forces/shocks between plunger rod 142 and plunger piston 144. The distal end 144a of the plunger piston 144 is configured to be movably displaced within the internal volume defined by the fluid container 150 by the energy released by the first energy storage member 134 when the first energy storage member 134 moves from its first configuration to second configuration. In this manner, the first energy storage member 134 acting on the plunger 140 can displace the plunger piston 144 proximally within fluid container 150 to expel the fluid through the orifice at proximal end 150b of fluid container 150.

The sidewalls of the proximal end 144b of the plunger piston 144 may include a piston plunger seal 146 (FIG. 3), configured to contact the interior surfaces of the sidewalls of the fluid container 150 such that the piston plunger seal 146 forms a fluid-tight seal with the sidewalls of the fluid container 150, for example, to prevent leakage of the fluid. The piston plunger seal can be made of an inert and/or biocompatible material. Example materials include rubber, silicone, plastic, polymers, any other suitable material or combination thereof. In some embodiments, the piston plunger seal 146 can be monolithically formed with the plunger 140. In some embodiments, the plunger 140 may also include one-way bendable tabs (not shown) to prevent movement of the plunger rod 142 in the distal direction once the plunger 140 has advanced to the second plunger position.

With continued reference to FIGS. 2 and 3, the delivery device 100 includes a driver 170. The driver 170 is shown as a cylindrical plug having a distal end 170a and a proximal end 170b and is sized and configured to be moveable in a proximal direction from a first driver position to a second driver position relative to lower body 120. The driver 170 further includes an inner cavity 171 sized and configured to support the needle 160. The inner cavity 171 further includes an expanded region 172 sized to accommodate a lower portion of fluid container 150.

The delivery device 100 also includes a second energy storage member 152. In the embodiment shown in FIGS. 2-7, the second energy storage member 152 is a mechanical energy storage member comprising a spring, such as, for example, a helical, compression, extension, torsion, constant, variable, variable stiffness or any other type of spring having a spring constant ranging between about 1 N/m to about 500 N/m. In an embodiment the second energy storage member is a spring having a spring constant of 370 N/m. The second energy storage member 152 is operatively coupled to the driver 170 and releases energy to the driver 170 to displace the driver 170 from its first driver position to its second driver position relative to lower body 120 when the second energy storage member 152 moves from its first configuration to its second configuration after the second releasable retainer member 122 releases the driver 170.

Referring to FIGS. 2 and 3, the delivery device 100 also includes a second releasable retainer member 122 which secures the lower body 120 to the driver 170 at a first driver position and is configured to release the driver 170 when the plunger 140 moves to the second plunger position. The second releasable retainer member 122 may be formed by one or more inwardly extending tabs formed in the surface of lower body 120 (FIG. 2), configured to engage corresponding openings 170c in the driver 170 when the second releasable retainer mechanism 122 is at the first position (FIG. 3). The second releasable retainer member 122, which may be what is commonly referred to as a snap (which may encompass a snap fit, snap joint snap lock or plastic clip) is inherently biased inwardly, so as to interferingly engage the opening 170c in the driver 170, such that a lower, inwardly protruding portion 122a of second releasable retainer member 122 engages the upper wall of opening 170c of the driver 170. With this arrangement, movement of the driver 170 relative to lower body 120 in the L direction is prevented. Once the plunger 140 has moved in a proximal direction from the first plunger position to the second plunger position, the second releasable retainer member 122 will then move from a first position to a second position, thereby releasing the lower body 120 from engagement with driver 170 (i.e., some of the energy released from the first energy member 134 is translated to the second releasable retainer member 122 when the plunger 140 is displaced to the second plunger position, the energy being sufficient to displace the second releasable retainer member 122 from a first position to a second position).

In some embodiments, delivery device 100 may further include a needle cover (not shown). The needle cover is configured to prevent needlestick injuries and maintain the sterility of the needle by providing a protective barrier around the needle. The needle cover can be manually removed before operation of the delivery device 100 or may be operable to automatically remove itself during the insertion of needle 160 into the subject then and re-cover the needle 160 after retraction of needle 160.

To illustrate operation of the delivery device 100, a sequence of events illustrating the injection and delivery of fluid by the device 100, as well as the position of the various components, is discussed progressing from FIGS. 3-6.

FIG. 3 illustrates the delivery device 100 in an initial position (i.e., first configuration), where the delivery device 100 is ready for use and activation. Upper body 110 is in a first upper body position. Actuator 130 is in the first actuator position. First and second energy storage members 134 and 152 are in their first configurations. The plunger 142 is held in place by the first releasable retainer member 132, the first releasable retainer member 132 being in engagement with the plunger 142 and the actuator 130. The driver 170 is at the first driver position and is cooperatively engaged with lower body 120. The needle 160 is fully within driver 170. Once the desired target site, such as an IM site (for e.g., the vastus lateralis muscle) is determined, the activation of the delivery device 100 can begin.

FIG. 4 illustrates the delivery device 100 in a second configuration. In one embodiment, a 2-step activation mechanism may be required to activate the actuator 130. First, the safety pin 112 is released from engagement and then the second safety activation mechanism which is configured to oppose the upward motion of the lower body 120 is released from engagement. In other embodiments, activation may be a 1-step activation where either the safety pin 112 or safety activation mechanism is present and must be released, while in still other embodiments the delivery device 100 does not include the safety pin 112 or safety activation mechanism. The delivery device 100 can be activated when force is applied at the proximal end 170b of the driver 170. This is typically the reaction force at the IM site as the user holds on to the upper body 110 and exerts force F upon the upper body 110 in the proximal direction. The upper body 110 is moved relative to lower body 120 in the proximal direction from the first upper body position to the second upper body position (as indicated by arrow A in FIG. 3). At this stage, plunger 140, fluid container 150 and needle 160 are coupled to upper body 110 and will also move in the proximal direction relative to lower body 120, such that a portion of needle 160 extends outside of the driver 170 to inject into the target site of the subject at a desired depth. Movement of upper body 110 moves actuator 130 in a distal direction relative to upper body 110 from the first actuator position to the second actuator position through contact of the distal end 120a of lower body 120 with the proximal end of actuator 130. As the actuator 130 moves to the second actuator position, the first releasable retainer member 132 is now able to release from the plunger rod 142. Energy is released from the first energy storage member 134 as it moves from its first configuration to second configuration, causing the plunger rod 142 to begin movement in the proximal direction within the chamber 114 and driving the plunger piston 144 towards proximal end 150b of fluid container 150 (as indicated by arrow B in FIG. 4). Thus, movement by the plunger rod 142 in the proximal direction towards second plunger position begins delivery of fluid from the fluid container 150 to the needle 160 and into the subject.

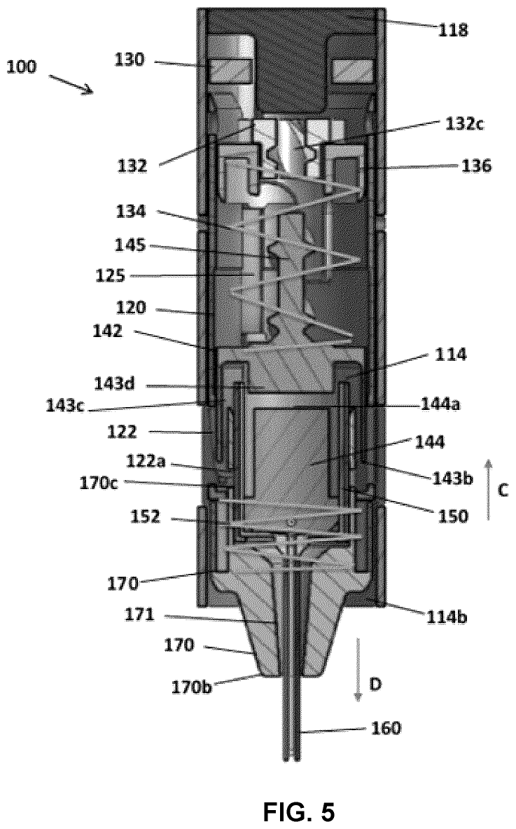

FIG. 5 illustrates the delivery device 100 in a third configuration. The plunger rod 142 has moved in the proximal direction to the second plunger position, driving plunger piston 144 to deliver substantially all of the fluid within the fluid container 150 to and through the needle 160 and into the subject. In this configuration, plunger rod 142 has moved over fluid container 150 such that fluid container 150 is partially received in inner cavity 143c of plunger rod 142. The proximal end 143b of the plunger rod 142 is now in cooperative engagement with the second releasable retainer member 122. Energy released by the first energy storage member 134 to the plunger rod 142 is transferred to the second releasable retainer member 122 in an amount that is sufficient to activate second releasable retainer member 122 and release the driver 170 from engagement with the lower body 120. In the embodiment shown in FIG. 5, the proximal end of plunger rod 142 pushes the tab of second releasable retainer member 122 outwards such that portion 122a is no longer in engagement with opening 170c of the driver 170. Thus, lower body 120 and driver 170 are able to move relative to each other.

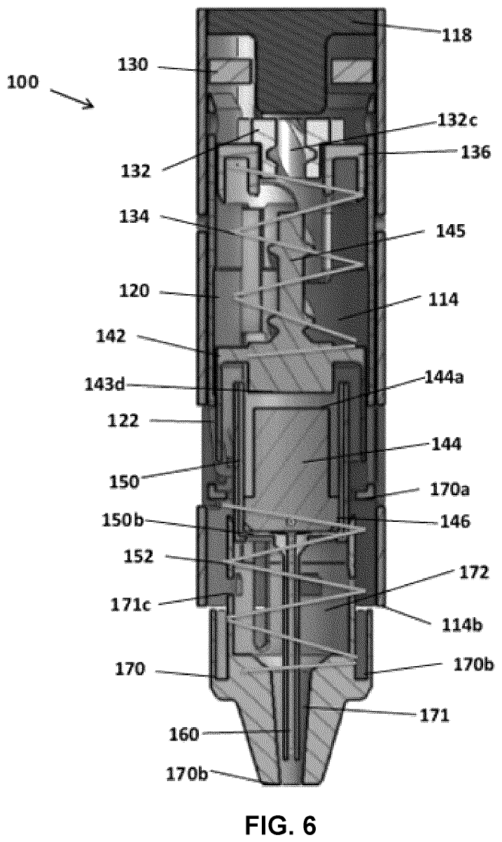

FIG. 6 illustrates the delivery device 100 in a fourth configuration. The second releasable retainer member 122 is activated and detaches the driver 170 from the lower body 120. With this the second energy member 152 moves from its first configuration to its second configuration. The distal end of the second energy storage member 152 is in contact with the proximal end 120b of the lower body 120 and the proximal end of the second energy member 152 is in contact with the driver 170. As described above for delivery device 1, the user may hold the device 100 to the target site for a prescribed time period, and after this prescribed time period, the user reduces the force F applied to the upper body 110. The second energy storage member 152 moving to the second configuration exerts force on the lower body 120 in the distal direction (as indicated by arrow C in FIG. 5) which in turn displaces upper body 110 in a distal direction which is sufficient, in combination with the reduced force F applied to the upper body 110 to displace delivery device 100 in a distal direction relative to the target site such that needle 160 is retracted from the target site. As this happens, driver 170 is displaced proximally (as indicated by arrow D in FIG. 5) by second energy storage member 152 relative to the needle from its first driver position (FIG. 5) to a second driver position (FIG. 6). As the driver 170 is displaced with respect to the lower body 120, the needle 160 is retracted back from the target site and is enclosed within driver 170.

Proximal movement of driver 170 may be restricted by the contact of a lip (not shown in FIGS.) on the upper portion of driver 170 contacting the inwardly extending proximal edge (not shown in FIGS. of the lower body 120 which prevents movement of driver 170 further than is shown in FIG. 6. The contact of the lip of driver 170 with the edge of lower body 120 is similar to as illustrated and described below with respect to delivery device 200.

In some embodiments, the delivery device 100 may also include a component for providing feedback to the operator once the injection and delivery of fluid is complete and the needle 160 is fully enclosed in the driver 170, such as, but not limited to, one or more viewing windows, such as windows 115a and 115b described above indicating that the injection and delivery of fluid has been completed or an audible click.

In some embodiments, needle 160 (and attached fluid container 150) of delivery device 100 may be driven by a force to drive the needle from a first needle position to a second needle position, whereby movement of the needle from the first needle position to the second needle position inserts the needle into the target site of the subject. In some embodiments, the force is an external force applied by a user to the delivery device, such as force F described above for delivery devices 1 and 100. In some embodiments, the force is applied by the first energy storage member 134 or the second energy storage member 152. In other embodiments, the force is applied by a third energy storage member (which may be similar to first and second energy storage members 134, 152 described above). The force may be applied to needle 160, fluid container 150 or other interconnected components. In an embodiment, the third energy storage member comprises a spring. For example, the third energy storage member may cause movement of needle 160 (and attached fluid container 150) in the proximal direction whilst a user holds the delivery device over the target site of a subject in order to drive the needle into target site of the subject. This may negate the need for a user to exert the force F on delivery device 100. The movement of needle 160 (and attached fluid container 150) may be initiated, for example, in response to the pressing of a button or switch on delivery device 100. An example of a suitable mechanism for driving needle 160 and fluid container 150 is disclosed in PCT application Serial No. PCT/CA2022/050057 filed on Jan. 14, 2022 and published on Jul. 21, 2022 as PCT publication no. WO 2022/150927 A1, the entire contents of which are incorporated by reference herein.

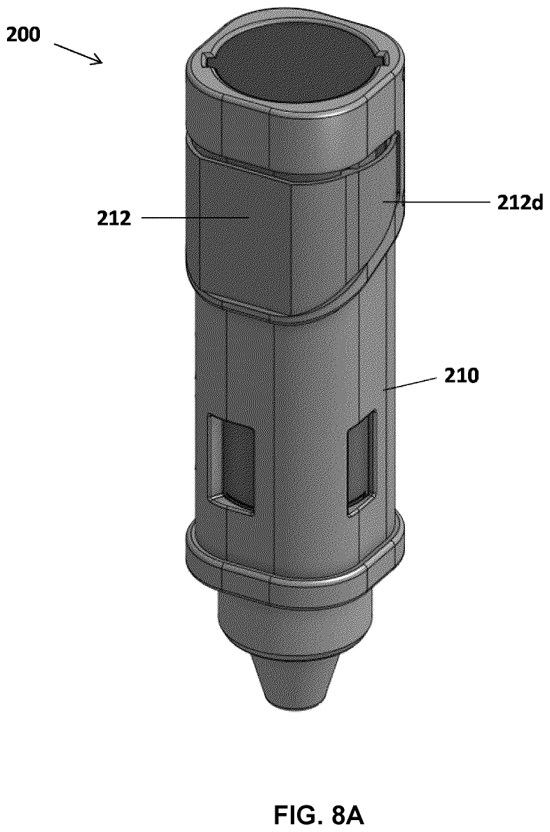

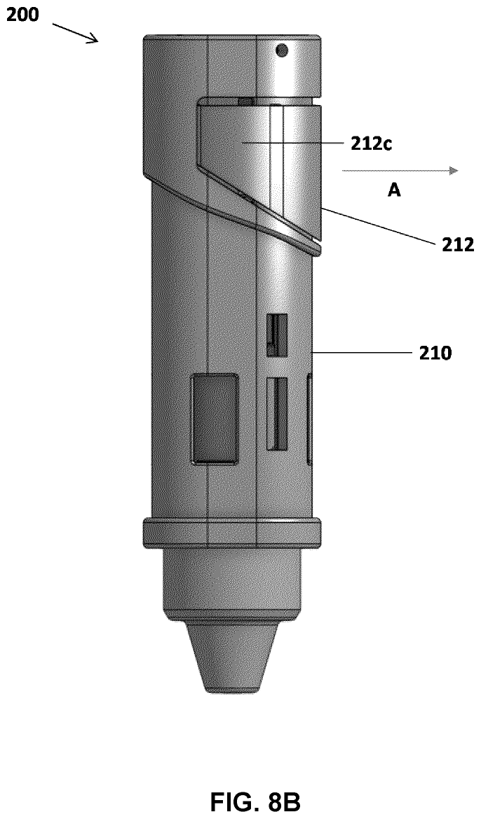

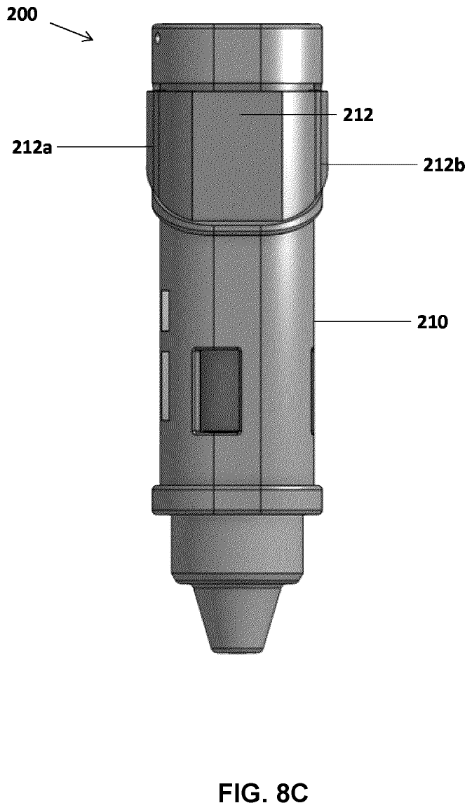

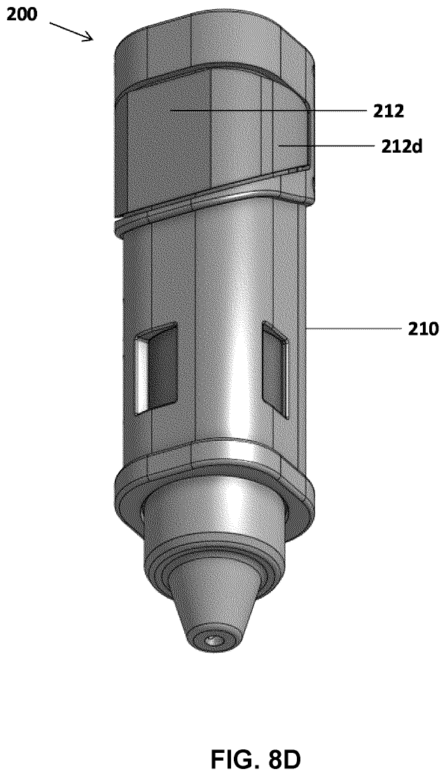

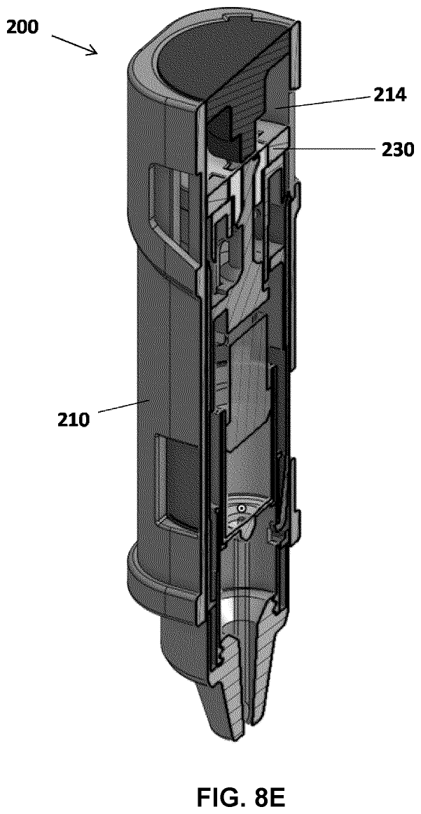

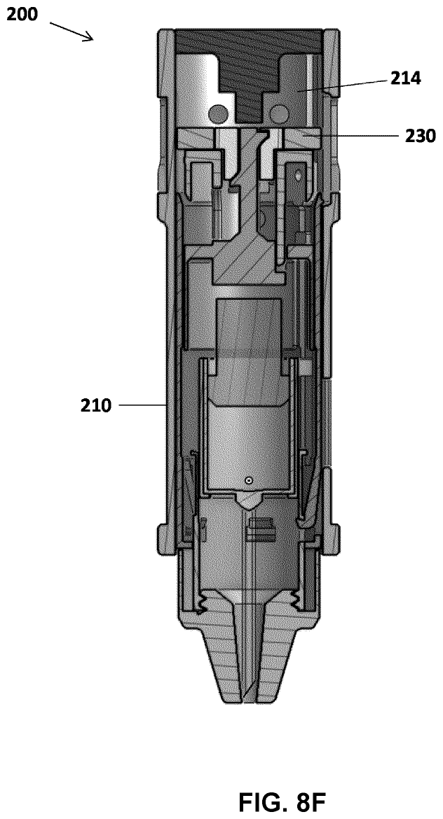

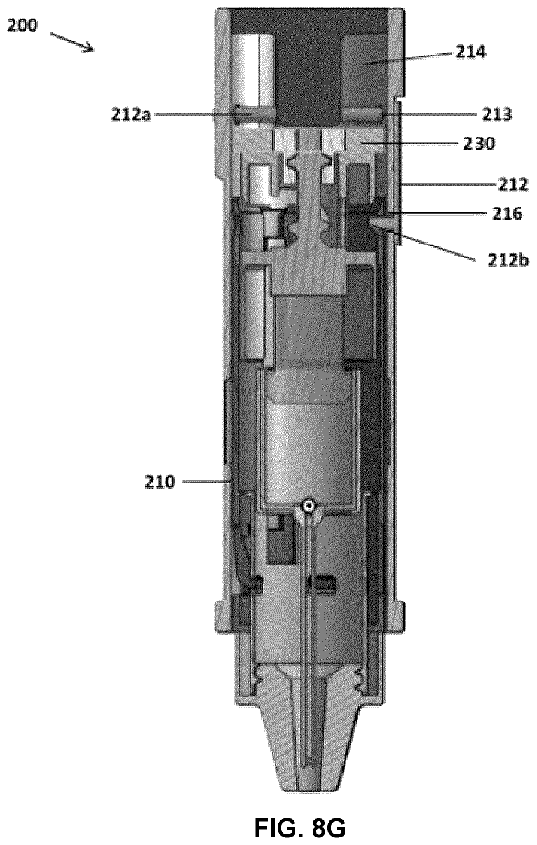

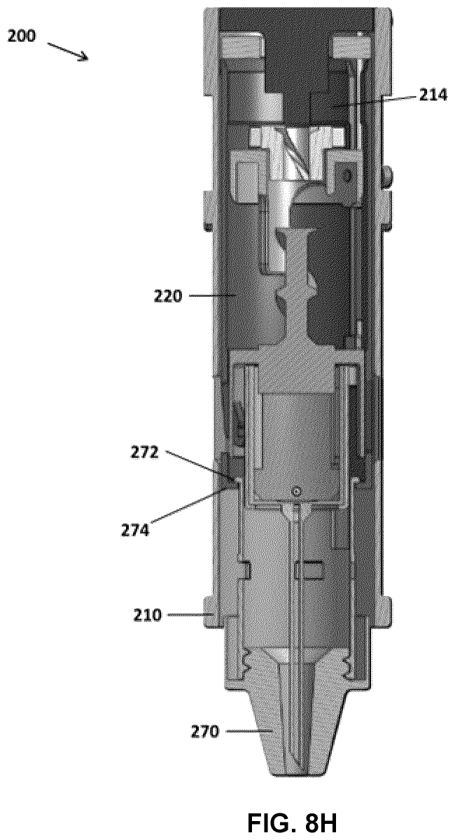

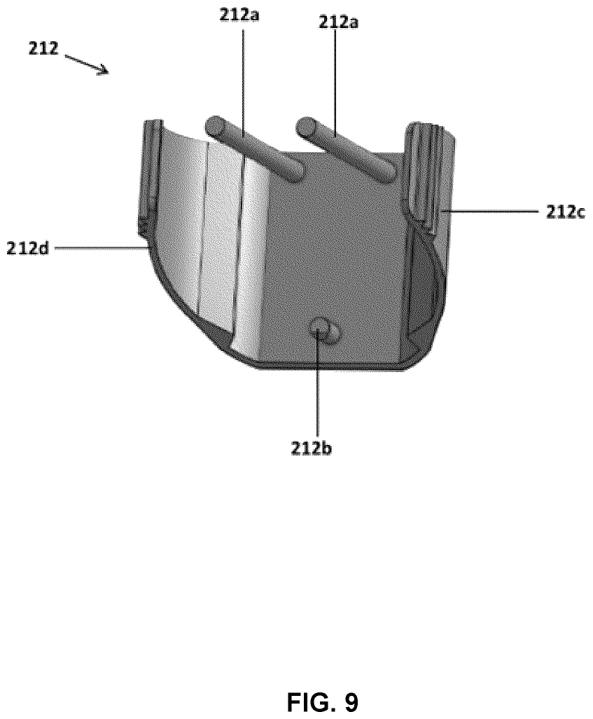

With reference now to FIGS. 8A-F a delivery device according to another embodiment is shown and generally designated by reference numeral 200. The delivery device 200 may generally be similar to delivery device 100 described above. In this embodiment, delivery device 200 includes an upper body 210 generally shaped and dimensioned to fit within an operator's hand. Delivery device 200 may also include a safety pin 212, which may operate in a similar manner to safety pin 112 described above. Safety pin 212, shown in isolation in FIG. 9, may include having one or more inwardly extending upper pins 212a and lower pin 212b configured to be inserted into respective openings 213, 216 (which may be similar to openings 113, 116 described above) in the upper body 210, as shown in FIG. 8G. Still referring to FIG. 8G, the pins 212a may extend into a chamber 214 of the device 200 and may perform a similar function to the upper portion 112a of the safety pin 112 described above, in this embodiment preventing movement of actuator 230 in a distal direction. Actuator 230 may be similar to actuator 130 described above. The safety pin 212 may be operated by a user gripping the opposed outer sides 212c, 212d of safety pin 212 and removing the safety pin 212 from the upper body 210 in the direction indicated by arrow A in FIG. 8B. This will also extract the pins 212a, 212b of the safety pin 212 through the slot 213, such that movement of the actuator 230 in a distal direction is no longer restricted by the safety pin 212. This will enable operation of the delivery device 200 in a similar manner to the delivery device 100.

With reference to FIG. 8H, delivery device 200 is shown with a driver 270 (which may be generally similar to driver 170 described above) in a second driver position, which may be similar to the second driver position of driver 170 described above. In this position, further proximal movement of driver 270 is prevented by engagement of lip 272 at the distal end of driver 270 with the inwardly extending proximal edge of lower body 220.

In another aspect, embodiments of the present disclosure are directed to a method for reducing or preventing hemorrhage, comprising intramuscularly administering an antifibrinolytic agent in a dose of about 0.1 g to about 30 g at a rate of no less than 50 mg/s; or use of an antifibrinolytic agent for reducing or preventing hemorrhage, wherein the agent is administered intramuscularly in a dose of about 0.1 g to about 30 g at a rate of no less than 50 mg/s. Rates described herein refer to average rates over the duration of injection. Without being limited by theory, it is believed that intramuscular injection of an antifibrinolytic agent at a rate of no less than 50 mg/s may facilitate the rapid absorption of the agent into the bloodstream of a bleeding patient. In some embodiments, the dose is administered at a rate of no less than 100 mg/s, no less than 150 mg/s, no less than 200 mg/s, no less than 250 mg/s, no less than 300 mg/s, no less than 330 mg/s, no less than 350 mg/s, no less than 400 mg/s, no less than 450 mg/s, or no less than 500 mg/s. In some embodiments, the dose is administered at a rate of about 250 mg/s. In some embodiments, the dose is administered at a rate of about 330 mg/s. In some embodiments, the dose is administered at a rate of about 220 mg/s to about 440 mg/s. In some embodiments, the dose is administered within about 1.1 seconds, about 1.6 seconds, about 2.2 seconds, about 5 seconds, within about 10 seconds, or within about 15 seconds. In some embodiments, the dose is administered within about 10 seconds. In some embodiments, the dose is administered within about 5 seconds. In some embodiments, the dose is administered within about 2.2 seconds. In some embodiments, the dose is administered within about 1.6 seconds. In some embodiments, the dose is administered in about 1.1 seconds to about 2.2 seconds.

In another aspect, embodiments of the present disclosure are directed to a method for reducing or preventing hemorrhage, comprising intramuscularly administering an antifibrinolytic agent with an autoinjector in a dose of about 0.1 g to about 30 g; or use of an antifibrinolytic agent for reducing or preventing hemorrhage, wherein the agent is administered intramuscularly with an autoinjector in a dose of about 0.1 g to about 30 g. Without being limited by theory, it is believed that intramuscular injection of an antifibrinolytic agent with an autoinjector achieves deeper penetration and better distribution of the agent than with a pre-loaded syringe. In some embodiments, the autoinjector is a multi-dose autoinjector.

In some embodiments, the antifibrinolytic agent comprises tranexamic acid (TXA) or F-aminocaproic acid (EACA). In some embodiments, the antifibrinolytic agent comprises TXA. In some embodiments, the antifibrinolytic agent comprises EACA.

In some embodiments, the dose is about 0.1 g to about 20 g, about 0.1 g to about 10 g, about 0.1 g to about 5 g, about 0.1 g to about 4 g, about 0.1 g to about 3 g, about 0.1 to about 2 g, about 0.1 to about 1.5 g, about 0.1 g to about 1 g, about 0.5 g to about 30 g, about 0.5 g to about 20 g, about 0.5 g to about 10 g, about 0.5 g to about 5 g, about 0.5 g to about 4 g, about 0.5 g to about 3 g, about 0.5 to about 2 g, about 0.5 to about 1.5 g, about 0.5 g to about 1 g, or about 1 g to about 2 g. In some embodiments, the dose is at least 0.1 g, 0.5 g, at least 1 g, at least 1.5 g, or at least 2 g. In some embodiments, the dose is about 0.1 g, about 0.2 g, about 0.3 g, about 0.4 g, about 0.5 g, about 0.6 g, about 0.7 g, about 0.8 g, about 0.9 g, about 1.0 g, about 1.1 g, about 1.2 g, about 1.3 g, about 1.4 g, about 1.5 g, about 1.6 g, about 1.7 g, about 1.8 g, about 1.9 g, about 2.0 g, about 2.5 g, about 3.0 g, about 3.5 g, about 4.0 g, about 4.5 g, about 5.0 g, about 6.0 g, about 7.0 g, about 8.0 g, about 9.0 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, about 25 g, about 26 g, about 27 g, about 28 g, about 29 g, or about 30 g. In some embodiments, the dose is about 1 g. In some embodiments, the dose is about 0.5 g.

In some embodiments, the dose is provided in an amount of fluid in the range of about 5 mL to about 20 mL, about 5 mL to about 10 mL, about 1 mL to about 20 mL, about 1 mL to about 10 mL, or about 1 mL to about 5 mL. In some embodiments, the dose is provided in about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL of fluid. In some embodiments, the dose is provided in about 10 mL of fluid. In some embodiments, the dose is provided in about 5 mL of fluid. In some embodiments, the dose is administered at a rate of no less than 0.1 mL/s, no less than 0.2 mL/s, no less than 0.3 mL/s, no less than 0.4 mL/s, no less than 0.5 mL/s, no less than 0.6 mL/s, no less than 0.7 mL/s, no less than 0.8 mL/s, no less than 0.9 mL/s, no less than 1.0 mL/s, no less than 1.1 mL/s, no less than 1.2 mL/s, no less than 1.3 mL/s, no less than 1.4 mL/s, no less than 1.5 mL/s, no less than 1.6 mL/s, no less than 1.7 mL/s, no less than 1.8 mL/s, no less than 1.9 mL/s, no less than 2.0 mL/s, no less than 1.1 mL/s, no less than 1.2 mL/s, no less than 1.3 mL/s, no less than 1.4 mL/s, no less than 1.5 mL/s, no less than 1.6 mL/s, no less than 1.7 mL/s, no less than 1.8 mL/s, no less than 1.9 mL/s, no less than 2.0 mL/s, no less than 2.1 mL/s, no less than 2.2 mL/s, no less than 2.3 mL/s, no less than 2.4 mL/s, no less than 2.5 mL/s, no less than 2.6 mL/s, no less than 2.7 mL/s, no less than 2.8 mL/s, no less than 2.9 mL/s, no less than 3.0 mL/s, no less than 3.1 mL/s, no less than 3.2 mL/s, no less than 3.3 mL/s, no less than 3.4 mL/s, no less than 3.5 mL/s, no less than 3.6 mL/s, no less than 3.7 mL/s, no less than 3.8 mL/s, no less than 3.9 mL/s, no less than 4.0 mL/s, no less than 4.1 mL/s, no less than 4.2 mL/s, no less than 4.3 mL/s, no less than 4.4 mL/s, no less than 4.5 mL/s, no less than 4.6 mL/s, no less than 4.7 mL/s, no less than 4.8 mL/s, no less than 4.9 mL/s, or no less than 5.0 mL/s. In some embodiments, the dose is administered at a rate of no less than 2.2 mL/s. In some embodiments, the dose is administered at a rate of no less than 3.3 mL/s. In some embodiments, the dose is administered at a rate of about 3.3 mL/s. In some embodiments, the dose is administered at a rate of about 2.2 mL/s to about 4.4 mL/s.

In some embodiments, the dose is administered at a fluid velocity of no less than 0.2 m/s, no less than 0.5 m/s, no less than 1 m/s, no less than 2 m/s, no less than 3 m/s, no less than 4 m/s, no less than 5 m/s, no less than 6 m/s, no less than 7 m/s, or no less than 8 m/s. “Fluid velocity” refers to the average volumetric flow rate divided by the cross-sectional area of the needle. In some embodiments, the dose is administered at a rate of no less than 4 m/s. In some embodiments, the dose is administered at a rate of no less than 4 m/s. In some embodiments, the dose is administered at a rate of no less than 6 m/s. In some embodiments, the dose is administered at a fluid velocity of about 4 m/s, about 5 m/s, about 6 m/s, about 7 m/s, or about 8 m/s. In some embodiments, the dose is administered at a fluid velocity of about 4 m/s to about 8 m/s.

As used herein, the administration or injection time refers to a duration starting from plunger release in an injection device and ending when substantially all of the fluid within the injection device is delivered to a subject. For example, when the delivery device 100 is used, the administration or injection time refers to the time from which the actuator 130 disengages from the first releasable retainer member 132 to the time at which the plunger 140 has advanced to the second plunger position, thereby delivering substantially all of the fluid in the fluid container 150 into the subject.

In some embodiments, the hemorrhage is posttraumatic hemorrhage, postpartum hemorrhage, or postoperative hemorrhage. In some embodiments, the hemorrhage is posttraumatic hemorrhage, e.g., hemorrhage caused by a traumatic brain injury. In some embodiments, the hemorrhage is postpartum hemorrhage. In some embodiments, the hemorrhage is postoperative hemorrhage. In some embodiments, the hemorrhage is external. In some embodiments, the hemorrhage is internal. In some embodiments, the hemorrhage is associated with hemorrhagic shock and/or coagulopathy. In some embodiments, the antifibrinolytic agent is administered within 5 hours, within 4 hours, within 3 hours, within 2 hours, within 1 hour, within 45 min, within 30 min, or within 15 min of the onset of the hemorrhage.

### Example: Injection Study on Swine Model

Fluid: TXA 100 mg/mL
Medium: Perfused swine tissue
Needle: 18G, 0.838 mm inner diameter
Spring: “90 N” nominal plunger spring

TABLE 1
Results from study on swine model

| | Volume | | | |
|---|---|---|---|---|
| | Injected | Injection | Average Flow | Average Fluid |
| | (mL) | Time (s) | Rate (mL/s) | Velocity (m/s) |
| mean | 5.055 | 1.538 | 3.355 | 6.083 |
| min | 4.902 | 1.190 | 2.280 | 4.134 |
| max | 5.208 | 2.150 | 4.376 | 7.935 |

The injection time refers to the time from which the actuator disengages from the first releasable retainer member to when the plunger has fully delivered substantially all of the fluid into the tissue. The calculated flow rates and linear fluid velocities inside the needle refer to average values for the duration of injection and are not indicative of instantaneous values. The results (see Table 1) show that a device of the present invention is capable of delivering a 5 mL dose of 100 mg/mL TXA within 1.6 seconds, and that average flow rates of at least 3.3 mL/s and average fluid velocities of at least 6.0 m/s can be achieved with a typical needle gauge.

While the foregoing is directed to embodiments of the present disclosure, other and further embodiments of the disclosure may be devised without departing from the basic scope thereof, and the scope thereof is determined by the claims that follow.

1. A delivery device operable to automatically inject and deliver a fluid to a target site of a subject, the delivery device comprising:
(i) a lower body;
(ii) an upper body configured to move relative to the lower body between a first upper body position and a second upper body position upon application of a force to the upper body; and
(iii) an injection assembly comprising:
(a) a fluid container configured to hold a fluid, the fluid container attached to the upper body and moveable with the upper body;
(b) a needle attached to the fluid container, the needle having a proximal end for insertion into the target site of the subject, wherein the delivery device is positionable such that the needle is inserted into the target site of the subject in response to the upper body moving from the first upper body position to the second upper body position;
(c) a plunger; and
(d) a first releasable retainer member configured to release the plunger in response to the upper body reaching the second upper body position, wherein the first releasable retainer member releasing the plunger causes the plunger to urge the fluid out of the fluid container and into the needle to inject the fluid into the target site through the proximal end of the needle when the proximal end of the needle is inserted into the target site.

2-48. (canceled)

49. A method for reducing or preventing hemorrhage, comprising intramuscularly administering an antifibrinolytic agent in a dose of about 0.1 g to about 30 g at a rate of no less than 50 mg/s.

50-62. (canceled)

63. A method for reducing or preventing hemorrhage, comprising intramuscularly administering an antifibrinolytic agent in a dose of about 0.1 g to about 30 g in about 1 mL to about 20 mL at a fluid velocity of no less than 0.2 m/s.

64-189. (canceled)

190. The delivery device of claim 1, wherein the injection assembly further comprises an actuator moveable relative to the first releasable retainer member from a first actuator position to a second actuator position in response to the upper body moving from the first upper body position to the second upper body position, wherein the first releasable retainer releases the plunger in response to the actuator moving to the second actuator position.

191. The delivery device of claim 190, wherein the actuator is configured to prevent rotation of the plunger relative to the first releasable retainer member when the actuator is in the first actuator position.

192. The delivery device of claim 1, wherein the first releasable retainer member is in engagement with the plunger through at least interlocking threads.

193. The delivery device of claim 1, wherein the delivery device further comprises a driver moveable relative to the lower body from a first driver position to a second driver position, wherein the driver is attached to the lower body when the driver is in the first driver position, wherein the driver moves proximally relative to the needle when the driver moves from the first driver position to the second driver position, wherein the driver comprises an inner cavity configured to house the needle when the driver is in the second driver position and the upper body is in the second upper body position.

194. The delivery device of claim 193, wherein the driver is attached to the lower body by a second releasable retainer member configured to secure the driver in the first driver position and release the driver to permit movement of the driver the second driver position in response to the plunger reaching the second plunger position.

195. The delivery device of claim 1, wherein the fluid container is configured to hold an amount of the fluid between about 1 mL and no more than about 10 mL.

196. The method of claim 49, wherein the antifibrinolytic agent is administered at a rate of no less than 100 mg/s, no less than 200 mg/s, no less than 300 mg/s, no less than 400 mg/s or no less than 500 mg/s.

197. The method of claim 49, wherein the antifibrinolytic agent is administered at a rate of about 250 mg/s or about 330 mg/s.

198. The method of claim 49, wherein the antifibrinolytic agent comprises tranexamic acid (TXA).

199. The method of claim 49, wherein the hemorrhage is posttraumatic hemorrhage, postpartum hemorrhage, or postoperative hemorrhage.

200. The method of claim 199, wherein the posttraumatic hemorrhage is a hemorrhage caused by a traumatic brain injury.

201. The method of claim 49, wherein the dose is about 0.5 g to about 2 g.

202. The method of claim 63, wherein the dose is provided in about 1 mL to about 10 mL of fluid.

203. The method of claim 63, wherein the antifibrinolytic agent is administered at a fluid velocity of no less than 0.5 m/s, no less than 1 m/s, no less than 2 m/s, or no less than 4 m/s.

204. The method of claim 63, wherein the antifibrinolytic agent is administered at a fluid velocity of about 6 m/s.

205. The method of claim 63, wherein the antifibrinolytic agent comprises tranexamic acid (TXA).

206. The method of claim 63, wherein the hemorrhage is posttraumatic hemorrhage, postpartum hemorrhage, or postoperative hemorrhage.

207. The method of claim 206, wherein the posttraumatic hemorrhage is a hemorrhage caused by a traumatic brain injury.

208. The method of claim 63, wherein the dose is about 0.5 g to about 2 g.
